(19) United States
(12) Patent Application Publication
(10) Pub. No.: US 2023/0305003 A1
(43) Pub. Date: Sep. 28, 2023

(54) AUTOMATED SAMPLE PREPARATION PLATFORM FOR CELLULAR ANALYSIS

(71) Applicant: Beckman Coulter, Inc., Brea, CA (US)

(72) Inventors: Gaelle Bouvier, Brea, CA (US); Andreas Boehmler, Brea, CA (US); David Faye, Brea, CA (US); Eduardo Flores-Fuentes, Brea, CA (US); Jesus Amundarain, Brea, CA (US); Daniel A. Flagler, Brea, CA (US)

(73) Assignee: Beckman Coulter, Inc., Brea, CA (US)

(21) Appl. No.: 18/014,194

(22) PCT Filed: Jul. 9, 2021
(86) PCT No.: PCT/US2021/041123

§ 371 Date: Jan. 3, 2023

Related U.S. Application Data
(60) Provisional application No. 63/050,637, filed on Jul. 10, 2020.

(51) Int. Cl. G01N 33/569; G01N 15/14

(52) U.S. Cl. CPC G01N 33/56972; G01N 15/1459; G01N 2015/1006

(57) ABSTRACT

The disclosure relates to, among other things, an automated flow cytometric method and system for the analysis and enumeration of at least one of hematopoietic stem cells, hematopoietic progenitor cells, and T-cells.

### CROSS-REFERENCE TO RELATED APPLICATIONS

This application is a national stage application of PCT/US2021/041123, filed Jul. 9, 2021, which claims the benefit of U.S. Provisional Appl. No. 63/050,637, filed Jul. 10, 2020, which is incorporated by reference as if fully set forth herein.

### BACKGROUND

Cellular analysis instruments using flow cytometers are known in the field. See, for example, Published U.S. Patent Application No. 2008/0010019, incorporated by reference as if fully set forth herein. A flow cytometer directs a flow of particles through a sensing zone where the particles can be excited by a beam of light. The beam of light causes the particles to fluoresce and/or scatter light, and the emitted light is separated by filters into portions of the electromagnetic (EM) spectrum. By studying the filtered EM spectrum, analyses of the cellular content can be performed and certain characteristics and values can be reported.

### SUMMARY

To date, however, no flow cytometers exist that allow for (i) the automated preparation of blood samples (e.g., umbilical cord blood samples) comprising hematopoietic stem cell, progenitor cell or T-cell samples, where, e.g., optimal incubation and volume details are preprogrammed; and (ii) the analysis and enumeration of at least one of the hematopoietic stem cells, hematopoietic progenitor cells, T-cells, and even leukocytes, in the same instrument where the samples are prepared. In addition, there are no flow cytometers that, in addition to (i) and (ii), allow for conducting analyses on single samples with or without a negative control; or replicate samples (e.g., duplicate samples) with or without a negative control. The instant disclosure describes such an instrument. The disclosure also provides a high-performing instrument, where the instrument's performance is improved, at least in part, by the ability to move various liquids (e.g., specimen types) with suspended solids (e.g., cells) accurately and precisely in order to enumerate the particles correctly.

Enumeration of CD34+ stem and progenitor cells is one of the most highly regulated tests in a clinical flow cytometry laboratory. There are four main factors that contribute to the criticality of this test:

(1) Before transplantation, donors usually undergo chemotherapy and/or radiotherapy that eliminate the patient's blood forming system, so that the recovery of the patient depends on the transplantation of a sufficiently large number of stem cells in order to reconstitute hematopoiesis. The correct enumeration of CD34+ cells is therefore mandatory.

(2) The regulatory frameworks for CD34+ enumeration differ by geography, however currently available quantification methods approved for IVD use do not provide the necessary flexibility to accommodate these differences, forcing laboratories to self-validate laboratory-developed tests on scarce and precious sample types in order to fulfill the requirements of their local governing bodies.

(3) Allogeneic (non-self) transplants contain residual immune competent CD3+ T cells that have the potential to cause Graft-versus Host Disease (GvHD), a potentially life-threatening complication in stem cell transplantation regimens. It is therefore mandatory to enumerate the number of CD3+ T cells in these samples, however there is currently no IVD kit on the market that allows for the parallel enumeration of CD34+ stem cells and CD3+ T cells in the sample, again forcing laboratories to work with user-defined tests.

(4) Currently available CD34+ numeration procedures are highly manual, causing potential room for human error and hence the need for sample re-runs, leading to delayed reporting of results or patient/donor discomfort in case additional samples need to be drawn. In addition, these samples often arrive as emergency (STAT) samples in the laboratory, disrupting the lab workflow and forcing lab staff to de-prioritize the analysis of other samples. Both factors led to the demand for an automated solution for CD34+ enumeration.

The newly developed AQUIOS STEM System and the methods described herein are the first in vitro diagnostic (IVD) solution for CD34+ enumeration that addresses these critical aspects. Based on the automated AQUIOS Flow Cytometry System and methods described herein, it provides a complete solution for automated CD34+ enumeration and optional CD3+ enumeration in a sample, resulting in a workflow that minimizes the need for human intervention. Samples are loaded on the system by the operator, and sample preparation and data analysis are performed automatically by the analyzer. This reduction of hands-on time provides a seamless workflow in the laboratory and minimizes the number of manual and hence potentially error-prone steps. This also means that CD34+ enumeration can be performed by non-flow cytometry experts, enabling laboratories to offer CD34+ enumeration outside regulatory lab office hours such as during night shifts or over the weekend. Both aspects increase the level of patient care and reduce time-to-result for this time-critical application.

In addition, AQUIOS STEM System and the methods described herein provide analysis options that are adapted to individual regulatory requirements, such as the International Society for Hematotherapy and Graft Engineering (ISHAGE) Guidelines for CD34+ enumeration and the European Pharmacopoeia, that differ in their request for duplicate runs and the use of a negative control. This flexibility also considers the parallel enumeration of CD3+ T cells together with the CD34+ stem and progenitor cell population, in that the AQUIOS STEM System provides a total of six (6) analysis options to choose from as part of the IVD solution. This is unique to the market and eliminates the need for time-consuming self-validation of lab-developed tests.

The level of automation provided by the AQUIOS STEM System is achieved by using pre-mixed and ready-to-use reagent combinations adapted for automation whilst ensuring the highest level of data traceability. Currently available manual test kits use solutions for red blood cell lysis (an essential step in sample preparation) that require manual daily preparation from a concentrate, and that negatively affect cell viability so that samples need to be stored on ice before the actual analysis. In contrast, AQUIOS STEM System uses a gentler red blood cell lysing reagent that is ready to use and can be used at room temperature, which allows automation of CD34+ and CD3+ counts in a way known for other automated flow cytometry applications, but not for stem cell enumeration. All reagent vials critical for the test are barcoded with individual identifiers and essential quality control parameters such as reagent type, reagent lot, day of first use, expiration date, etc., and stored in one database together with the sample information, providing the level of data traceability mandated by today's accreditation authorities.

AQUIOS STEM System is validated in clinical studies against its predicate method, the Stem-Kit for the FC500 flow cytometer, that is perceived as the “gold standard” in the market for clinical CD34+ enumeration and that was also used as reference for the manual CD34+ enumeration solutions of other manufacturers. With the AQUIOS STEM System, the “gold standard” is taken to the next level by providing a unique new solution for the enumeration of CD34+ stem and progenitor cells that takes away the burden of stem cell enumeration from clinical laboratories.

### BRIEF DESCRIPTION OF THE DRAWINGS

These and other features, aspects, and advantages of the present technology will become better understood with regard to the following drawings:

FIG. 1A is a checklist of the characteristics of a solution for the identification and quantification of CD34+ hematopoietic progenitor cells that addresses requirements of clinical laboratories.

FIG. 1B is a flow diagram of a method described herein for the analysis and enumeration of at least one of hematopoietic stem cells, hematopoietic progenitor cells, and T-cells.

FIG. 2 is a perspective view of one example of a diagnostic instrument, wherein the instrument is shown coupled with a specimen autoloader and includes a flow cytometer.

FIG. 3 is an enlarged perspective view of a portion of the diagnostic instrument shown in FIG. 2.

FIG. 4 is a front perspective view of the diagnostic instrument of FIGS. 2-3, showing the instrument during operation.

FIG. 5 is an enlarged view of the portion of the diagnostic instrument that is capable of sampling a single specimen tube at a time.

FIG. 6 is a front perspective view of the external housing of the proposed diagnostic instrument shown in FIGS. 2-5.

FIG. 7 is a front perspective view of the external housing of another example, in which the specimen autoloader is removed and specimen tubes are inserted through the front door.

FIG. 8 is an example of a software component system that can be used in the methods and systems described herein.

FIG. 9 is a table showing the panel options for the clinical quantification of CD34+ HPC, either with or without the analysis of residual T cells.

FIGS. 10A-10C are plots showing equivalence of the three CD34+ analysis options STEM Panel (tests in duplicate plus negative control), STEM Duplicate (tests in duplicate, no negative control) and STEM Single (single test) for CD34+ absolute counts (cells/μL). These three analysis options are for tests that do not include CD3.

FIGS. 11A-11C are plots showing the equivalence of the three CD34+ plus CD3+ analysis options STEM ALLO Panel (tests in duplicate plus negative control), STEM ALLO Duplicate (tests in duplicate, no negative control) and STEM ALLO Single (single test) for CD34+ absolute counts (cells/μL). These three analysis options are for tests that do include CD3.

FIGS. 12A-12C are plots showing the equivalence of the three CD34+ plus CD3+ analysis options STEM ALLO Panel (tests in duplicate plus negative control), STEM ALLO Duplicate (tests in duplicate, no negative control) and STEM ALLO Single (single test) for CD3+ absolute counts (cells/μL). These three analysis options are for tests that do include CD3.

FIG. 13 is a representative manual flow cytometry workflow, as described in Example 2.

FIG. 14 is a representative AQUIOS CL Flow Cytometry System workflow, as described Example 2.

FIGS. 15A-15B are plots of sample processing turnaround time and operator hands-on time for one CD34+ sample or a batch of 10 CD34+ samples on the FC500 with Stem-Kit (Alternative Method, empty bars) and AQUIOS STEM System (AQUIOS, hashed bars).

FIGS. 16A-16B are plots of process time and operator hands-on time for Quality Control (QC) procedures on the FC500 with Stem-Kit (Alternative Method, grey bars) and AQUIOS STEM System (AQUIOS, red bars) per workday (FIG. 16A) and a 5-day work week (FIG. 16B).

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

With the increased use of mobilized, peripheral stem and progenitor cells (PBSC) for transplantation purposes, researchers, in concert with the International Society for Hematotherapy and Graft Engineering (ISHAGE), described in 1996 a set of standards for CD34+ enumeration with the intent to provide a simple, sensitive method that allows for a high degree of accuracy and interlaboratory reproducibility. The resulting “ISHAGE Guidelines” soon became the gold standard for the enumeration of hematopoietic CD34+ progenitor cells by flow cytometry. In 1998, a research group published a modified version of the 1996 guidelines, by introducing beads for absolute counting, adding 7-aminoactinomycin D (7-AAD) as viability dye to exclude dead cells, and a lysing reagent lacking fixatives, such as formaldehyde. These modifications converted the basic protocol into a single-platform method, and the resulting “Single platform with viability dye ISHAGE Guidelines” are still, more than 20 years later, mostly untouched.

The hallmark of the ISHAGE Guidelines is a sequential gating strategy that derives the number of CD34+ cells from viable leukocytes. The Guidelines also require tests to be run in duplicate together with a negative control in order to correct for testing variability and nonspecific cell and fluorochrome binding.

Due to the selectivity of the sequential gating, the ISHAGE Guidelines made the use of a negative control optional. In contrast, the European Pharmacopoeia (Ph. Eur.) mandates the use of a control. In contrast to the ISHAGE Guidelines, the Standards described in the Ph. Eur. are legally binding, as laid down in the Council of Europe Convention on the Elaboration of a European Pharmacopoeia and in EU and national pharmaceutical legislation.

Currently available software solutions that are part of an in vitro diagnostic (IVD) system do not provide the flexibility to run panels for CD34+ enumeration either with or without a negative control while maintaining IVD status. In addition, not all CD34+ enumeration reagent kits contain a negative control reagent.

While the starting point of the sequential ISHAGE gating strategy tremendously facilitates the correct quantification of rare CD34+ cell populations, it leads to the fact that as an end point all enumerated CD34+ cells are automatically viable, and it can be difficult to directly calculate the percentage of viable CD34+ cells out of all CD34+ cells. In addition, it can be challenging to retrieve the total number of CD45+ leukocytes from the applied gating strategy in order to assess overall specimen viability.

For CD34+ cells derived from cord blood, the recently released 7th Edition of the “NetCord—FACT International Standards for Cord Blood Collection, Banking, and Release for Administration” requires the determination of both the total CD34+ count as well as the total viable CD34+ count for cord blood samples post-processing prior to cryopreservation, and the assessment of percent viability of CD34 prior to release of a cord blood unit to the clinical program.

In case CD34+ quantification is performed for quality control purposes, laboratories seek the ability to not run a “full” ISHAGE panel consisting of duplicate tests plus negative control, but rather a single test, especially when only small specimen volumes are available for analysis. Current acquisition software does not provide the flexibility to adapt the panel accordingly as part of the IVD solution. Although for QC purposes an IVD system is not necessarily required, most laboratories performing these tests are highly regulated and therefore refrain from validating a separate user defined test only for this purpose.

In cases of hematological diseases or non-malignant dysfunction of the hematopoietic system, CD34+ cells are collected from the peripheral blood (PB), bone marrow (BM) or cord blood (CB) of non-self (allogeneic) donors. Like in autologous settings, where the donor and the recipient are the same person, mobilized PB today is the most commonly used source for CD34+ stem and progenitor cells in allogeneic transplant schemes.

The success of an allogeneic hematopoietic progenitor cell (HPC) transplant depends on a variety of factors, such as the availability of a suitable donor, human leukocyte antigen (HLA) compatibility, successful balancing of the patient's immune response while maintaining the Graft versus Leukemia/Tumor effect of the transplant, and others. The use of CD34+ cells from mobilized peripheral blood has the advantage of a relatively rapid recovery of hematopoiesis after transplantation but goes along with an increased risk of acute Graft versus Host Disease (aGvHD) due to a higher number of circulating T-cells. As acute and chronic GvHD affects approximately 30-40% of patients who undergo allogeneic transplantation, and donor T cells are recognized as playing a central role in mediating aGvDH, the enumeration of CD3+ T-cells in the graft together with CD34+ cells has become standard practice in many laboratories. As there is currently no commercially available kit that allows for the IVD analysis of CD34 and CD3 in one test, laboratories again need to rely on the validation of user defined tests for this application.

Laboratories performing CD34+ HPC enumeration are highly regulated in terms of data traceability and need to establish an extensive QC system, especially when undergoing accreditation. Some key aspects of these control mechanisms include (a) the avoidance of sample misidentification throughout the process by adequate identification of all samples; (b) adequate provisions for monitoring the reliability, accuracy, precision, and performance of test procedures and instruments; (c) functional checks for instruments and reagents; (d) the use of appropriate reference material and the documentation of ongoing proficiency testing; (e) a process to prevent the use of expired reagents and supplies; and (f) a mechanism that allows to link the lot number, expiration date, and manufacturer of supplies and reagents to each specimen.

Especially aspects (e) and (f) can be challenging, as the documentation of reagents and specimens are often not interlinked and happen “offline”, such as not on the platform used for data acquisition and analysis.

In hemato-oncological laboratories, HPC samples often arrive as emergency (STAT) samples in the laboratory and require immediate attention, disrupting the routine workflow. Any issues with these samples duplicate efforts and thus increase the potential for human error. For these laboratories, it would be desirable to integrate HPC samples into the normal workflow, ideally in a way that minimizes the risk for sampling mistakes or other issues, as the analysis of CD34+ HPC is time critical. For laboratories that are specialized in CD34+ enumeration, such as cord blood facilities, a higher degree of automation would help to manage the increasing number of samples to be analyzed, while providing a high standard of traceability as outlined herein.

Current IVD solutions for CD34+ enumeration by flow cytometry lack automation capability, mainly due to the use of red blood cell lysing reagents that negatively affect cell viability so that prepared samples need to be kept on ice prior to analysis. The ISHAGE Guidelines suggested the use of ammonium chloride, because it basically was the only lysing reagent available at the time that was suited for a lyse/no wash approach without the need for additional fixatives, and without altering scatter properties of the cell population of interest. While the use of ammonium chloride is well established in basically all commercial CD34+ enumeration kits for flow cytometry, it prevents the implementation of CD34+ tests on automated sample preparation and flow cytometry platforms due to its effect on cell viability and because the working dilution needs to be prepared fresh daily.

Today ready-to-use fixative-free red blood cell lysing reagents are available that specifically lyse red blood cells without pronounced impact on leukocyte viability during sample preparation, allowing to perform CD34+ enumeration on an automated flow cytometry system.

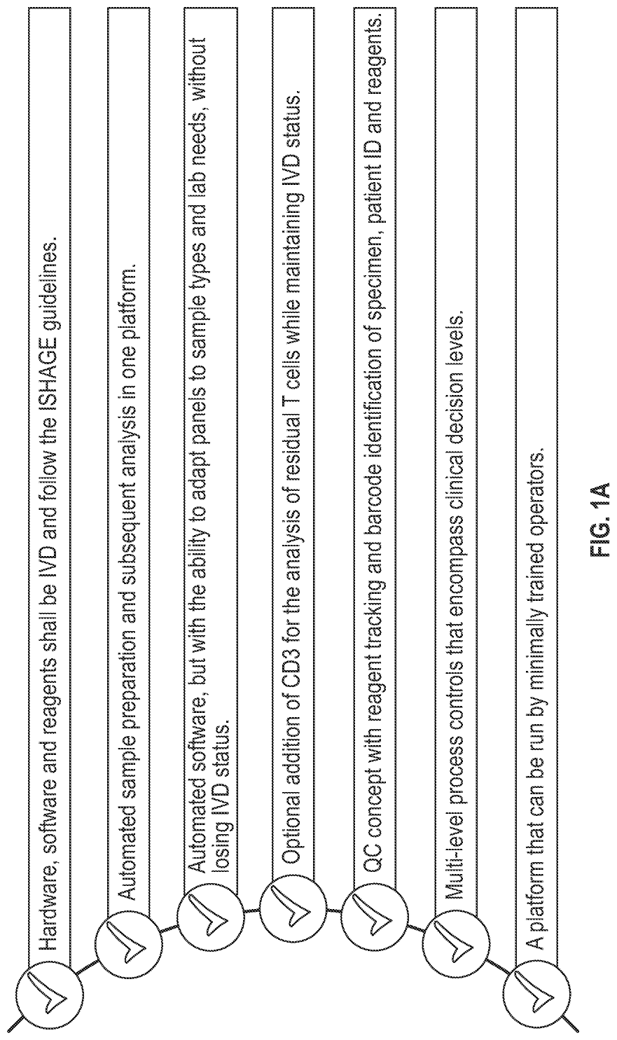

The ideal CD34+ quantification kit for flow cytometry combines the benefits of established standards and protocols with enough flexibility to adapt reagent and software tools to the needs of a clinical laboratory. This includes acquisition and analysis panels for different sample types without having to set up user defined tests in parallel to IVD solutions, quality control mechanisms that meet the requirements of a highly regulated work environment, and a high degree of automation. See FIG. 1A.

The methods described herein for the analysis and enumeration of at least one of hematopoietic stem cells, hematopoietic progenitor cells, and T-cells, were designed with the goal bringing the so-called Gold Standard to the “next level.” The methods described herein embody a modular approach for the automated analysis of, among other things, CD34+ hematopoietic stem and progenitor. In one example, the methods described herein include software and reagent kits for CD34+ enumeration; software and reagent kits for the simultaneous enumeration of CD3+ T cells and CD34+ cells in sample material from allogeneic donors; and CD34 control cells (2 Levels), as described in Tables 1 and 2 herein.

TABLE 1

| | Software | Tests | |
|---|---|---|---|
| | Panel Option | per panel | |
| STEM KIT | Run tests in | 1 | CD45-FITC/CD34- |
| CD45-FITC/CD34-PE | duplicate plus | | PE/7-AAD |
| CD45-FITC/CD34-Ctrl | negative control | 2 | CD45-FITC/CD34- |
| 7-AAD | | | PE/7-AAD |
| Lysing reagent | | 3 | CD45-FITC/CD34- |
| Beads for absolute | | | CTRL/7-AAD |
| counting | Run tests in | 1 | CD45-FITC/CD34- |
| | duplicate | | PE/7-AAD |
| | | 2 | CD45-FITC/CD34- |
| | | | PE/7-AAD |
| | Single test | 1 | CD45-FITC/CD34- |
| | | | PE/7-AAD |

TABLE 2

| | Software | Tests | |
|---|---|---|---|
| | Panel Option | per panel | |
| STEM Kit + STEM Allo- | Run tests in | 1 | CD45-FITC/CD34- |
| CD3 Kit | duplicate plus | | PE/CD3-PC7/ |
| CD45-FITC/CD34-PE | negative control | | 7-AAD |
| CD45-FITC/CD34-Ctrl | | 2 | CD45-FITC/CD34- |
| 7-AAD | | | PE/CD3-PC7/ |
| Lysing reagent | | | 7-AAD |
| Beads for absolute | | 3 | CD45-FITC/CD34- |
| counting | | | CTRL/CD3-CTRL/ |
| CD3-PC7 | | | 7-AAD |
| CD3-CTRL | Run tests in | 1 | CD45-FITC/CD34- |
| | duplicate | | PE/CD3-PC7/ |
| | | | 7-AAD |
| | | 2 | CD45-FITC/CD34- |
| | | | PE/CD3-PC7/ |
| | | | 7-AAD |
| | Single test | 1 | CD45-FITC/CD34- |
| | | | PE/CD3-PC7/ |
| | | | 7-AAD |

wherein “CD45-FITC” generally refers to fluorescein-conjugated antibody, permitting the analysis and enumeration of cell populations expressing the CD45 antigen present in human biological samples using flow cytometry, manufactured by Immunotech SAS (a Beckman Coulter Company), Marseille, France; “CD34-PE” generally refers to phycoerythrin-conjugated antibody permitting the analysis and enumeration of cell populations expressing the CD34 antigen present in human biological samples using flow cytometry, manufactured by Immunotech SAS (a Beckman Coulter Company), Marseille, France; “CD3-PC7” generally refers to phycoerythrin cyanin 7-conjugated antibody permitting the analysis and enumeration of cell populations expressing the CD3 antigen present in human biological samples using flow cytometry. Although fluorophores such as FITC, PE, and PC7 have been specified herein, one can use any suitable fluorophore-conjugated antibodies for that permit the analysis and enumeration of cell populations expressing the CD45, CD34 antigen, and the CD3 antigen, so long as the fluorophore can be detected within the detection capabilities of the instrument.

The methods described herein can use CD34 control cells, which are liquid preparations of stabilized human leukocytes for the verification of the parameters CD34, CD45 and CD3 as part of the systems described herein. In one example, kits contain two levels of CD34 with approximately ten CD34+ cells/μL (Level 1) and approximately 30 CD34+ cells/μL (Level 2). Assay values can be entered into the system by scanning a barcode of a control cell assay sheet.

As used herein, the term “hematopoietic stem cells” or “HSC” generally means cells having both pluripotency, which allows them to differentiate into functional mature cells such as granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), and monocytes (e.g., monocytes, macrophages), and the ability to regenerate while maintaining their pluripotency (self-renewal).

As used herein, the term “hematopoietic progenitor cells” or “HPCs” generally means cells having the potential to differentiate into functional mature cells such as granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), and monocytes (e.g., monocytes, macrophages).

HSCs and/or HPCs are optionally obtained from the body or an organ of the body containing cells of hematopoietic origin. Such sources include un-fractionated bone marrow, umbilical cord, and peripheral blood. All of the aforementioned crude or un-fractionated blood products can be enriched for cells having hematopoietic stem cell characteristics in ways known to those of skill in the art.

During hematopoiesis, HSCs first diverge into the progenitor stage into the myeloid lineage and the lymphoid lineage, then differentiate into myeloid stem cells (mixed colony forming cells, CFU-GEMM) and into lymphoid stem cells, respectively. Further, myeloid stem cells differentiate into erythrocytes via erythroid burst forming cells (BFU-E) and erythroid colony forming cells (CFU-E), into thrombocytes via megakaryocyte colony forming cells (CFU-MEG), into monocytes, neutrophils and basophils via granulocyte-macrophage colony forming cells (CFU-GM), and into eosinophils via eosinophil colony forming cells (CFU-Eo), while lymphoid stem cells differentiate into T-cells via T lymphoid progenitor cells and into B cells via B lymphoid progenitor cells. These myeloid stem cells and various hematopoietic progenitor cells derived from them are identified by the properties of colonies they form on soft agar, semisolid methylcellulose media or the like in the presence of various cytokines.

In the example shown in Table 2, for the analysis of residual CD3+ T-cells in samples of allogeneic origin, an optional allogenic-CD3 kit can be combined with the base kits described in Table 1. The combination of both kits provides a validated solution for the combined quantification of CD34 and CD3 in one run, with the ability to choose between the three panel options described herein.

With this framework in mind, the disclosure relates to an automated flow cytometric method, such as the one shown in FIG. 1B, for the analysis and enumeration of at least one of hematopoietic stem cells, hematopoietic progenitor cells, and T-cells, the method comprising:
placing precise volumes of one or more blood samples (e.g., blood aliquots from the same patient blood sample) into one or more receptacles of a sample plate located in a sample preparation area of a flow cytometer;
treating the one or more receptacles with at least one reagent to obtain at least one first composition, at least one of the at least one reagent being at least one imaging reagent comprising recognition elements specific for markers on at least one of the hematopoietic stem cells, the hematopoietic progenitor cells, and T-cells;
incubating the at least one first composition for a time period sufficient for binding of the at least one imaging reagent comprising recognition elements specific for the markers on the at least one of the hematopoietic stem cells, hematopoietic progenitor cells, and T-cells to give at least one second composition;
treating the at least one second composition with a lysing reagent to give at least one third composition; and
analyzing the at least one third composition by flow cytometry to obtain at least one of a viable and total hematopoietic stem cell count viable and total progenitor cell count, and viable and total T-cell count in the one or more blood samples. The method can further comprise performing a precise addition of counting beads, then analyzing the at least one third composition by flow cytometry to obtain at least one of a viable and total hematopoietic stem cell count viable and total progenitor cell count, and viable and total T-cell count in the one or more blood samples. The first composition can further comprise a viability dye as an example of the at least one reagent. As discussed herein, one example of a viability dye is 7-aminoactinomycin D (7-AAD).

As discussed herein, the methods described herein can be conducted with duplicate blood aliquots from the various sources described herein. Alternatively, or in addition, the methods described herein can be conducted with a negative control (or not). See, e.g., the Table 1 and Table 2 test panels. The negative control can be for CD34. Alternatively, the negative control is for CD3. And as described herein, the markers are at least one of CD45 (e.g., the CD45 antigen expressed on certain cell populations, including hematopoietic stem cells), CD3 (e.g., the CD3 antigen expressed on certain cell populations, including T-cells), and CD34 (e.g., the CD34 antigen expressed on certain cell populations, including hematopoietic stem cells). The negative control can be an isotype control or an isoclonic control. In an isoclonic control, cells are stained in the presence of an excess of identical unlabeled antibody. The unlabeled antibody takes up all the binding sites, preventing the labeled antibody from binding specifically. Thus, any signal that is detected must come from non-specific binding.

The at least one imaging reagent can be any suitable imaging agent, including imaging agents comprising a fluorescent reporter. Thus, for example, the imaging agents contemplated herein can be antibodies conjugated with a fluorescent reporter, including, but not limited to, FITC, PE, PC7, and the like.

The methods described herein can provide at least one of the viable and total hematopoietic stem cell counts, viable and total progenitor cell counts, and viable and total T-cell counts.

The methods described herein also include an automated flow cytometric method for the analysis and enumeration of at least one of hematopoietic stem cells, hematopoietic progenitor cells, and T-cells, the method comprising: placing a first blood sample into a first receptacle of the sample plate;
treating the first receptacle with at least one reagent to obtain a first composition f1 and incubating first composition f1 to give second composition s1, at least one of the at least one reagent being at least one imaging reagent comprising recognition elements specific for markers on at least one of the hematopoietic stem cells, hematopoietic progenitor cells, and T-cells;
while first composition f1 is incubating, placing a second blood sample into a second receptacle of the sample plate and treating the second receptacle with at least one reagent, at least one of the at least one reagent being at least one imaging reagent comprising recognition elements specific for markers on at least one of hematopoietic stem cells, hematopoietic progenitor cells, and T-cells, to obtain a first composition f2 and incubating first composition f2 to give second composition s2;
treating second compositions s1 and s2 with a lysing reagent to give third compositions t1 and t2; and
analyzing third compositions t1 and t2 by flow cytometry to obtain at least one of a viable and total hematopoietic stem cell count, viable and total progenitor cell count, and viable and total T-cell count.

In some instances, as described herein, the methods can further comprise further preparing a negative control. The negative control can be prepared before the first composition f1; the negative control can be prepared after the first composition f1; or the negative control can be prepared after the first composition f2.

The systems and methods described herein have various advantages. For example, the systems and methods described herein streamline operations by incorporating automated loading, sample preparation, reagent management, and barcode scanning and patient tracking, as well as data analysis and bidirectional LIS connectivity in one platform. Further, the systems and methods described herein allow for the addition of CD34+ enumeration to the existing software, so that most steps of the methods described herein can be automated. This is achieved by the use of a red blood cell lysing reagent that fulfills the criteria of the ISHAGE Guidelines (e.g., no wash, no addition of fixatives, no alteration of scatter characteristics of the target population), but can be stored and handled at room temperature, is ready to use without the need for daily preparation of a working dilution from stock solutions, and is gentle to the populations of interest. Examples of suitable lysis reagents include, but are not limited to the VersaLyse Lysing Solution manufactured by Immunotech SAS (a Beckman Coulter Company), Marseille, France. See, e.g., U.S. Pat. No. 7,30,797, which broadly describes lysis reagents and incorporated by reference as if fully set forth herein.

The beads solutions used for absolute counting (see Tables 1 and 2) have a carefully balanced buoyancy that does not require individual mixing before each pipetting step. Also, the bottle that contains the beads comes with a special cap that avoids evaporation.

Samples are loaded either using the cassette autoloader or the single tube loader for STAT samples that are then prioritized over other samples in a queue. This ensures a seamless workflow without the need to interrupt ongoing processes. Sample preparation can take place in 96 well deep-well plates, or any suitable sample plate, where the samples are not only prepared, but where samples are also treated in a treating step that is performed by an automated pipettor configured to deliver predetermined volumes of the at least one reagent. Sample preparation can take place with individual probes for sample preparation and analysis, so that both steps happen in parallel and samples are analyzed as soon as sample preparation is complete.

The reagents used in the systems and methods described herein comprise a unique barcode identity for tracking the expiration date, on board expiration, lot, and container numbers. The reagent consumption and plate usage are monitored by the systems described herein as the samples are processed. The bar code reader scans a reagent or plate upon first use, ensuring that “consumable” information is error free or at least substantially error free. The systems described herein consider reagent containers or plates to be at full capacity the first time they are “seen” by the system. The systems described herein can comprise a reagent tracking system, such as the AQUIOS Smart Track reagent tracking system present in the AQUIOS CL flow cytometer (Beckman Coulter Life Sciences, Indianapolis, IN), provides real time consumable tracking to ensure that reagents with proper dating are used, and that there is sufficient reagent for each sample; this eliminates the risk of having to re-run samples because of insufficient reagents levels or missing reagents. For example, the systems described herein will not run a test unless it finds the necessary antibody vials.

The systems described herein have additional advantages. For example, the systems described herein can have a tube barcode reader that can automatically read specimen barcodes without the use of a handheld barcode scanner. The system matches the barcode on the tube to an LIS request. The specimen ID is recorded before specimen aspiration to prevent misidentification, and the specimen ID is automatically tracked throughout the run.

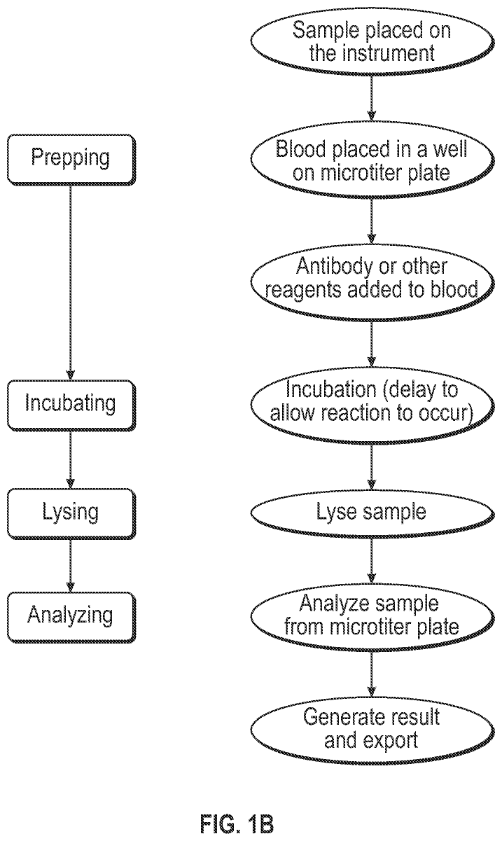

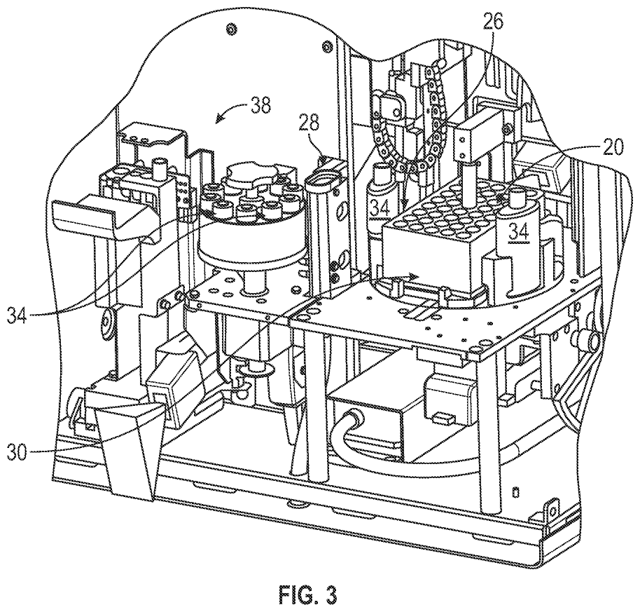

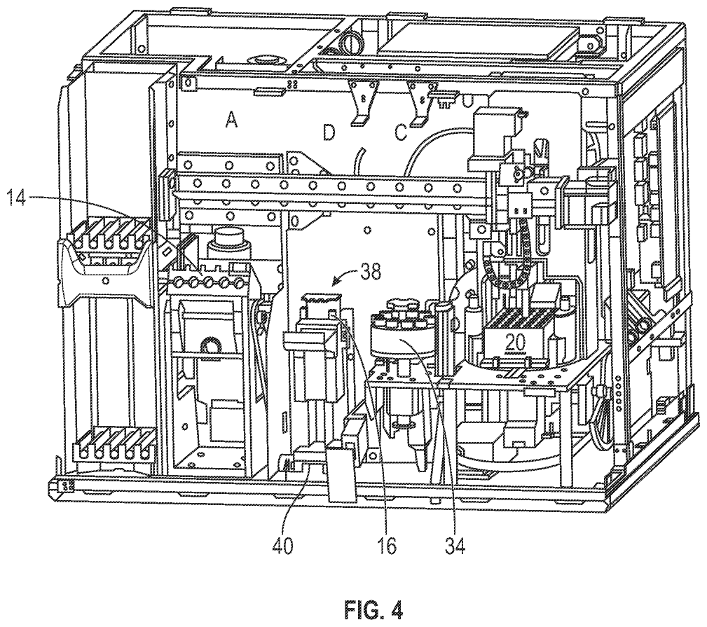

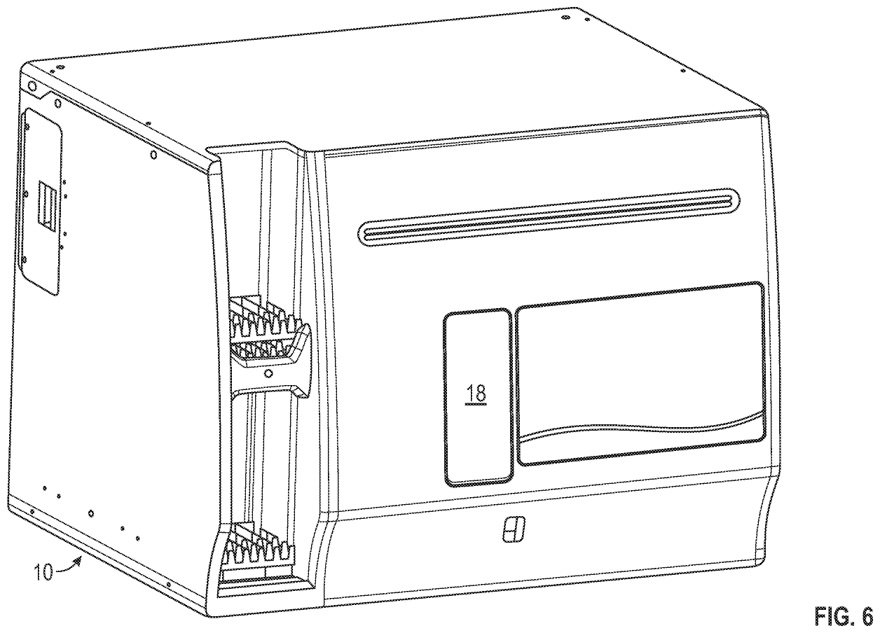

An example of a system that can carry out the methods described herein includes the system shown in FIGS. 2-7 in the form of a diagnostic instrument 10. In the illustrated example, an autoloader portion 12 can be seen having a number of specimen cassettes 14 loaded thereon. In such an example, cassettes 14 can be loaded with a plurality of identical specimen tubes or vials (hereinafter referred to as “tubes”) 16, a variety of specimen tubes 16, or merely a single specimen tube 16. The cassettes are then top-loaded into autoloader portion 12 and processed in the order received. In the alternative, e.g., when faster, single-sample processing is desired, a specimen tube can be inserted directly into an alternative specimen entry point, e.g., door 18 (visible in FIG. 5) and processed ahead of any awaiting cassettes 14, as shown in FIG. 4. This provides for STAT access to the testing by a clinician, with a capability to run tests immediately, thereby interrupting (but not negatively affecting) testing of other specimen tubes when desired by the clinician. Additionally, a specimen tube that has compromised or no bar coding (discussed infra) can be inserted manually.

As described in detail herein, diagnostic instrument 10 illustratively performs the following steps once a specimen tube 16 (or specimen tube cassette 14) is received. It is contemplated that such steps are performed by instrument 10 without intervention by a clinician, and the steps can be modified, added to, or eliminated depending on the particular test(s) to be performed. It should be understood that while blood tubes are discussed throughout the disclosure, it is contemplated that other types of body fluids and samples are within the scope of the disclosure, and capable of being analyzed in the proposed instrument 10. For example, bone marrow, serum, urine, synovial, spinal, peritoneal, plural, and other types of fluids or samples can be tested and analyzed substantially as described below.

The steps that can be performed by instrument 10 include: mixing (e.g., rocking) samples while still in specimen tubes 16 (e.g., while in an autoloader); piercing the cap of specimen tubes 16 and sampling the requisite amount of the specimen; reading barcodes (or any other form of marking/identifying) to confirm sample/patient ID and/or to confirm type/size of tube; matching ID, test(s) to be performed, and reagents required, and assigning a serial number for tracking by the computer; placing the specimen/sample in selected empty tubes or wells in a containment area 20 (shown, for example, as a microtiter plate in FIGS. 2-4), for further processing; adding appropriate reagents in the appropriate sequence, including mixing of the reagents once added, and timing so as to properly prepare the samples for the tests to be performed; allowing the samples to react with reagents for prescribed incubation times (variable based on the reagent); splitting the sample into a plurality of tubes/wells in the containment area 20 (if desired or required by testing); tracking all samples, cassettes, reagents and relevant positions via barcodes or other type of tracking device (e.g., RFID); timely aspirating the prepared sample/reagent combination from the containment area and analyzing it via flow cytometer (while preparing subsequent samples; auto-verifying results or holding results for review, depending on clinician-initiated decision rules.

Instrument 10 provides, among other things automated and integrated specimen sampling, meaning that each of the above steps (if required by the particular tests) can be carried out within and by instrument 10, without the use of additional diagnostic equipment. Moreover, if desired by the clinician, such steps can be done without any interaction from the clinician. It should be understood, however, that instrument 10 can be configured to alert a clinician in the event of a fault or other problem.

In the illustrated example, instrument 10 uses a single-axis probe carrier 22 that permits various functions to be performed while probe carrier 22 is moved along single-axis track 24. For example, probe carrier 22 (and therefore probe 26) can be positioned to draw samples from tubes 16 when probe carrier 22 is in position A, can deposit the samples in containment area 20 at position B, and can sample reagents at position C. If a sample is placed in pivotable tray 36 at any point (e.g., for STAT processing of a sample), instrument 10 can sense the presence of the sample and insert it ahead of any samples awaiting processing in the autoloader 12. Probe carrier 22 can then move to position D so that probe 26 can sample from the tubes placed in pivotable tray 36. Reagents are deposited in containment area 20 either before or after the sample is deposited (or both before and after) for reaction with the sample as required by the particular test(s) to be performed and can be themselves tracked as discussed herein.

The steps can be performed in the following order. However, it is contemplated that certain tests may skip one or more steps, or may modify a step in order to achieve the best test results for the desired blood test(s).

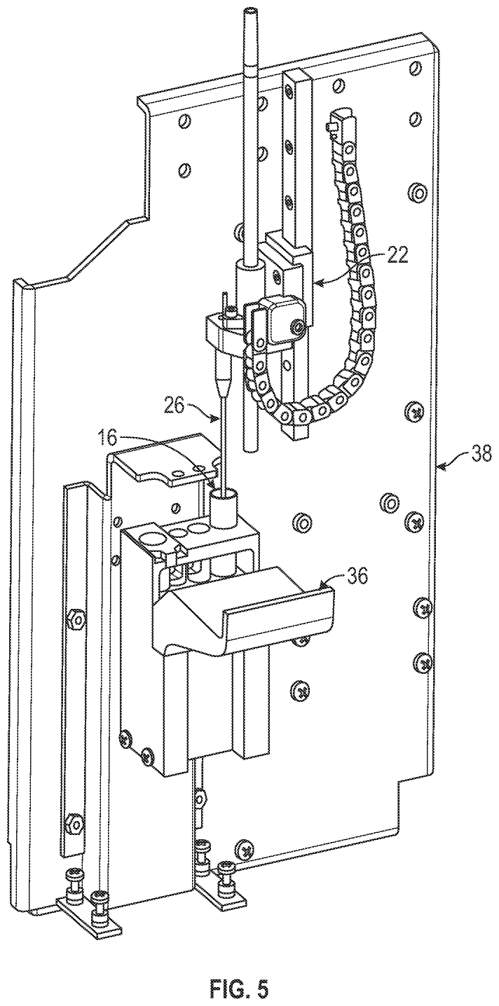

First, specimen tubes 16 can be loaded into a pre-configured cassette 14 that is appropriate for the particular specimen tubes 16 to be used. For example, specimen tubes 16 can be a commonly found size of 13 mm×75 mm specimen tube, in which case the five-tube cassette 14 shown in FIGS. 2 and 4 can be used. However, it should be understood that a variety of sizes and types of specimen tubes 16 can be used, and cassettes 14 can be designed accordingly. A cassette 14 can even be configured to hold a variety of specimen tubes 16. As stated herein, various sizes of specimen tubes 16 may also be inserted individually through door 18, shown in FIG. 6.

If specimen tubes 16 have a cap 32, the specimen tubes (held by cassette 14) can be rocked such that the blood is stirred inside the tube and made more homogenous (for more accurate sampling). Such rocking can occur at station A, and cassette 14 can be seen in its rocked position in FIG. 4.

During rocking of cassette 14, probe carrier 22 can be directed to move to station C and begin sampling the appropriate portions of reagents 34 for the tests to be performed. However, if the test does not contemplate reagents 34 being placed on the containment area 20 prior to the blood sample, then probe carrier 22 may perform such step after sampling the blood from tube 16.

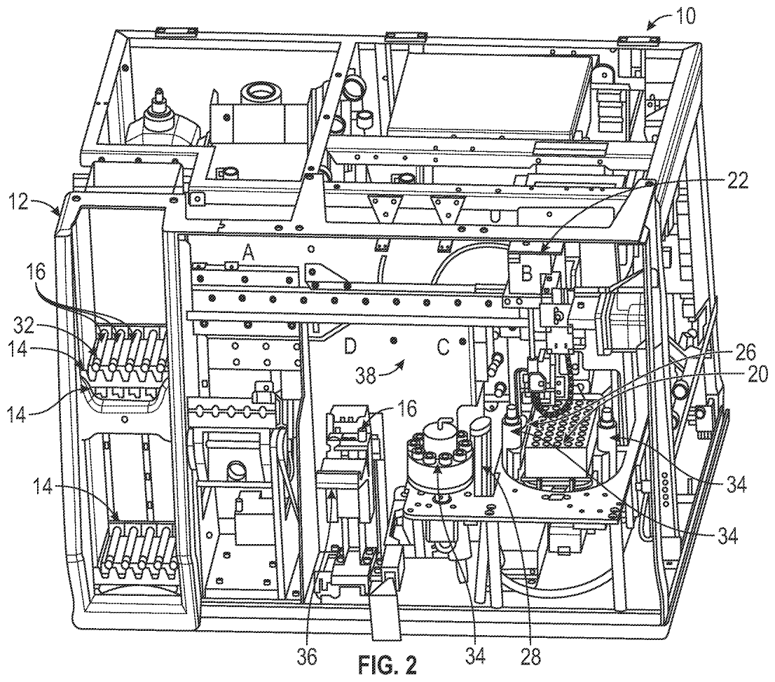

Reagents 34 (e.g., the reagents described in Tables 1 and 2) can be held in vials, as can be seen at position C. However, reagents can alternatively or additionally be held in reservoirs positioned elsewhere, such as on the plate base 30 (shown in FIGS. 2 and 3), or in other areas (not visible) that can be, for example, plumbed directly to probe 26.

As set forth herein, diagnostic instrument 10 also contemplates that a clinician can insert a specimen tube 16 via external door 18. To accommodate this, a tube receiver 38 is provided in illustrated instrument 10, and such tube receiver can accommodate a variety of types of specimen tubes 16, including pediatric tubes, as can be seen in FIGS. 3-5. In the illustrated example, specimen tubes 16 can be held by a pivotable tray 36 that permits easy access and retrieval of specimen tubes 16. Alternatively, as shown in FIG. 4, specimen tubes 16 can be held by a rotatable cassette 40.

In between and after sampling of specimens and/or reagents 34, probe carrier 22 can move to a probe washer station 28, so that probe 26 can be washed. Washing the probe 26 prevents cross-contamination and therefore prevents inaccurate test results.

After sufficient mixing is done of the specimen within the tubes (e.g., at station A), the specimen is sampled by probe 26 and deposited in predetermined wells or tubes in containment area 20. Depending on the test(s) to be performed, such as the analysis and enumeration of at least one of hematopoietic stem cells, hematopoietic progenitor cells, and T-cells using the methods described herein, specimen samples can be placed in more than one well or tube, and the corresponding amount of specimen (such as blood) can be aspirated in advance. Probe 26 is then washed at washer station 28 as described herein.

Depending on whether or not reagents are added to the specimen samples after depositing them in containment area 20, the probe carrier 22 can be moved to station C for sampling of the appropriate reagent(s) 34. Again, if more than one reagent is needed, probe 26 can be washed at washer station 28 between each reagent 34 sampling and after the final reagent 34 sampling.

In order to deposit specimen samples and reagents in each well or tube of containment area 20, plate base 30 can be positioned on a rotating axis so that each well or tube could be presented to probe 26, depending on the point of rotation of the plate base 30. Such a configuration and rotational movement of plate base 30 is disclosed in U.S. Pat. No. 7,832,292, which is incorporated herein by reference.

Although a multi-axis probe carrier can also accomplish these goals, certain advantages exist for a single-axis device. For example, a single axis device requires fewer parts and less programming, yields a smaller instrument 10 footprint, is easier to align, is more reliable and more stable, and ultimately faster in its movement between stations.

After placement in wells or tubes, the specimen samples are left to react with reagents for a specific amount of time (depending on the reagents and the tests to be performed) and then processed through the flow cytometer for analysis. It is contemplated that other test equipment can also be incorporated, such as equipment that uses electronic volume for cell sizing and differentiation, or hemoglobin measurement using absorbance.

Conveniently, containment area 20 serves as a common interface between sample preparation and analysis. Moreover, containment area 20 can include fixed or detachable, and/or disposable or reusable components, allowing a clinician to opt to throw away the entire interface (e.g., a microtiter plate) after use. By serving as the common interface between the preparation arm and analysis arm, containment area 20 provides a system with less exposure to mistakes and external or environmental influences.

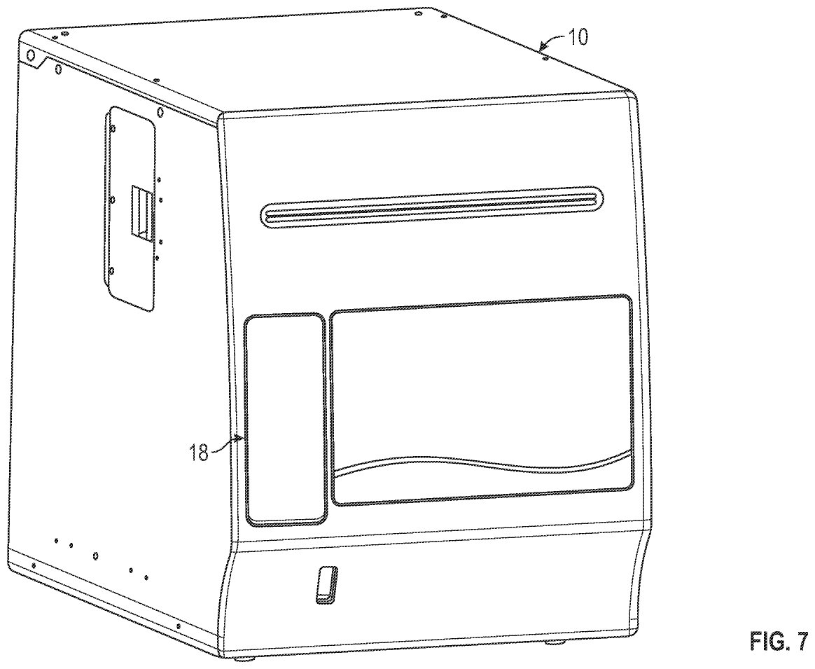

A processor and software scheduler configured to run on the processor (not shown) are also incorporated in the disclosed system. The software scheduler can be programmed, for example, to recalculate available windows for fixed reaction kinetics (optimizing throughput while maintaining reproducible reaction kinetics) (e.g., antibody incubations, RBC lysing time, reaction quench time, etc.). An example of a software component system that can be used in the methods and systems described herein is shown in FIG. 8.

Also, as discussed herein, numerous items can be barcoded and tracked during operation. Such barcoding and tracking can be registered by the software scheduler. For example, barcodes can be assigned to the reagent vials 34, specimen tubes 16 (with different bar codes for different patients and/or sizes), sheath fluids, common interfaces (e.g., containment areas 20), preparation reagents, bead reagents, cassettes 14, etc. By bar coding these various items, a variety of significant information can be tracked, such as reagent usage/consumption, how many tests remain for each reagent bottle, open container expiration, closed container expiration, assay values, etc.

The software scheduler can be configured to perform the steps described in FIG. 8 and/or the following steps: decide if it is ok to add a new sample at this time or not, and hold off door or multi-loader (random access) if another activity needs to take precedence; minimize the sample door 18 unavailable effect by adjusting non-kinetic reactions if any, or kinetic reactions that have a broader acceptable window; minimize collision effects, and optimize throughput by defining acceptable windows for each kinetic reaction; force analysis to take a predetermined amount of time (stop on time/fixed volume of sample); use predetermined times for each cycle, (acquiring of blood, adding of reagents including mixing, analysis) so all activities can be properly scheduled; take all scheduled sample time windows into effect in determining if it is acceptable to add a new sample to the schedule, and schedule such new sample so that all its activities take place at the predetermined times; and take hardware resources and physical hardware collisions into effect in determining if the schedule can be accomplished.

Using instrument 10 in combination with the software scheduler disclosed herein, a Time to First Result (TFR) can be less than an hour, with subsequent results reported about every 30 minutes or less. Throughput can be more than 5, more than 10, more than 20, more than 30, more than 40, more than 50, more than 60, more than 70, more than 80, more than 90 or more than 100 samples per day, and results can be reported much quicker and earlier in the day, so a laboratory's capacity can be significantly increased.

In the illustrated example, the analysis of data for obtaining reportable results is automated (e.g., setting of gates, regions, and cursors as well as flagging or notification of suspect results). The flagging/notification aspect can be referred to as an auto-verify feature in the system.

With all sample preparation and analysis fully integrated in one instrument 10, a clinic need not perform slow and tedious “batch processing,” where the samples are collected and processing started once a sufficient number are collected—progressing through each step of the blood processing with the entire group of samples. In contrast, instrument 10 is configured to automatically prepare patient samples in containment area 20, so there are no daughter tubes to label and keep track of, and significantly less blood and reagents are needed. Samples can be loaded onto the system at any time, and in one example, each will be automatically processed and exit the system pipeline in less than an hour, such as in less than 30 minutes, less than 20 minutes or less than 15 minutes. Subsequent samples could exit the system pipeline in approximately 30 minute or less intervals, although exact times will vary depending on the tests to be performed and required sample preparation times.

A significant advantage is the cost savings for a lab. Not only can more samples be processed in a single day, by using one system, there are lower system costs, lower reagent costs and reduced hands-on labor. Accordingly, the overall cost to own and operate the instrument 10 is significantly lower.

In addition, prior art processes and systems, with their multiple modules and computer screens, take up between 10 and 13 feet of valuable bench space. In contrast, diagnostic instrument 10 is compact, measuring only 31 inches wide, inclusive of the autoloader portion 12. The example shown in FIG. 6, without an autoloader portion, requires an even smaller footprint. A touch-screen computer/screen (not shown) can also be conveniently placed on top of the system, keeping the footprint small and freeing up valuable space for the lab.

It is contemplated that the proposed system can also be used for clinical researchers running one or more fixed immune surveillance panels for contract research, pharmaceutical drug development, and research in university medical centers and reference labs. It is further contemplated that in addition to the described tests for stem and T-cell analysis and enumeration, existing standardized immune monitoring panels can be used for monitoring immunodeficiency (HIV-AIDS), autoimmune diseases, organ transplant response, infectious diseases, oncology and others. Both applications can be run on the system in parallel.

In general, two characteristics contribute to performance: resolution (the ability to measure two particles with the same quantity of fluorescence and assign them the same value); and sensitivity (ability to differentiate between a dim particle and a slightly brighter particle). To measure these characteristics, “microspheres” or “beads” can be used herein. These microspheres can be made from, for example, fluorophore-labeled materials that have known fluorescence values. When such microspheres are passed through a flow cytometer, certain tests have been performed that reflect the resolution and sensitivity values being measured by the flow cytometer.

The bead test can be followed by another test to ensure that the reagents used are performing properly. Users with training in the art of flow cytometers make determinations—largely based on experiences or their own (variable) insights—as to whether the flow cytometer was sufficiently “optimized” such that it could perform the required diagnostic tests that day.

The diagnostic tests to be performed by a flow cytometer can have different minimum resolution and sensitivity needs than the bead and reagent tests that are dominant in the industry. It can be the case that the bead and reagent tests will indicate to a clinician that the flow cytometer is not optimized, whereas in reality, the flow cytometer is operating sufficiently to perform the diagnostic tests required—it simply did not pass the hypothetical bead and reagent tests.

Therefore, it can be desirable to utilize a known patient sample, for example a blood processing control, so that any deficiency in resolution or sensitivity can be narrowed down to the reagents and instrument performance. When a known patient sample is used, for example a blood control, only the reagents and instrument performance can affect the resolution and sensitivity outcomes of the test.

IN some instances, a known patient sample can be used as a control sample/initial test sample. The known patient sample is characterized as having distinguishable populations, e.g., at least two types of cells, that are either similar or identical to the populations to be diagnosed by that particular instrument. In the example of a diagnostic device that will be performing standardized immune monitoring panels, such as that shown in FIGS. 2-7, the known sample will have a cell content that includes the types of cells (e.g., CD34+ hematopoietic stem cells) to be analyzed by instrument 10.

According to this example, once the known patient sample is run through an instrument 10 undergoing evaluation, the results should indicate whether instrument 10 was able to detect a plurality of populations of cells. If the resolution and sensitivity of the instrument are optimized, distinct populations of cells should be indicated in the results. Software can be used to calculate off light scatter, ECV and/or fluorescence data in the manners described herein.

The disclosed example can also be used to derive a statistic to measure the resolution and sensitivity of an instrument 10 for a particular parameter on a particular test and quantify it in terms of sufficiency for running the test. Such a statistic may then be used to determine whether materials used in the test are adequate. For example, the statistic can be used to define the minimum resolution and sensitivity needs from the cytometer/reagent package, and then analyze whether the cytometer/reagent package is performing adequately to run the test on any patient. The statistic may also be used to determine if data from a previous patient sample should be accepted as accurate. The end result may also be a numeric means of qualifying performance for a test.

### EXAMPLES

### Example 1

Enumeration of CD34+ hematopoietic progenitor cells and residual CD3+ T cells with the system described herein: Verification of test protocol equivalence

### Background

With the increased use of mobilized, peripheral stem and progenitor cells (PBSC) for transplantation purposes, Sutherland et al., in concert with the International Society for Hematotherapy and Graft Engineering (ISHAGE), described in 1996 a set of standards for CD34+ enumeration with the intent to provide a simple, sensitive method that allows for a high degree of accuracy and interlaboratory reproducibility. The resulting “ISHAGE Guidelines” soon became the gold standard for the enumeration of hematopoietic CD34+ progenitor cells by flow cytometry.

The hallmark of the ISHAGE Guidelines is a sequential gating strategy that derives the number of CD34+ cells from viable leukocytes. The Guidelines also require tests to be run in duplicate together with a negative control in order to correct for testing variability and unspecific cell and fluorochrome binding. Due to the selectivity of the sequential gating, the use of a negative control was made optional by the authors of the ISHAGE Guidelines in subsequent years, however the European Pharmacopoeia (Ph. Eur.) mandates its use. In contrast to the ISHAGE Guidelines, the Standards described in the Ph. Eur. are legally binding, as laid down in the Council of Europe Convention on the Elaboration of a European Pharmacopoeia and in EU and national pharmaceutical legislation.

Currently available software solutions that are part of an IVD system do not provide the flexibility to run panels for CD34+ enumeration either with or without a negative control while maintaining IVD status. In addition, not all CD34+ enumeration reagent kits contain a negative control reagent. Laboratories are therefore required to validate their own user-defined tests.

In case stem and progenitor cells are transplanted in an allogeneic (non-self) setting, it is recommended to not only quantify the number of CD34+ progenitor cells, but also the number of immune competent CD3+ residual T cells in the graft in order to predict and manage a potential graft-versus-host disease. As of today, there is no commercial IVD kit available that allows for the simultaneous analysis of CD34+ progenitor cells and CD3+ residual T cells, so that laboratories again need to rely on the validation of user defined tests for this application.

### AQUIOS STEM System

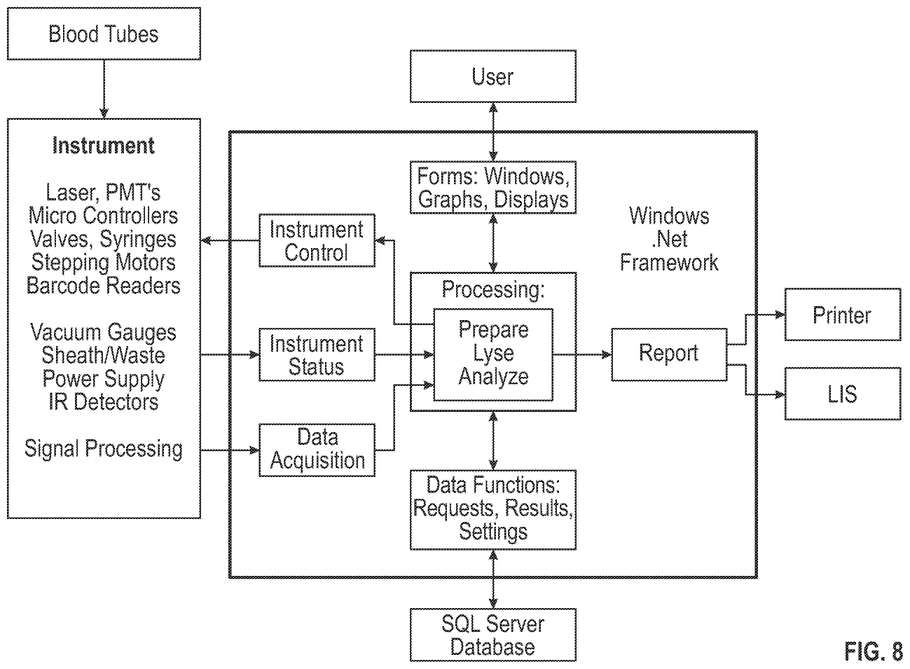

The system described herein is intended to, among other things, overcome these limitations by providing a total of six (6) different acquisition panels for clinical CD34+ enumeration (FIG. 9). All protocols follow the sequential gating strategy of the ISHAGE Guidelines, and the panels provide the option to either run the “full” panel of three tests (duplicate plus negative control) as suggested by ISHAGE and mandated by the Ph. Eur., the optional ISHAGE panel without the use of a negative control, or a single test that can be used for QC purposes for rare specimen types. All panel combinations are part of the IVD solution without the need to create user defined tests.

For the analysis of residual CD3+ T cells in samples of allogeneic origin, the optional AQUIOS STEM Allo-CD3 Kit can be combined with the base AQUIOS STEM Kit. The combination of both kits provides a validated solution for the combined quantification of CD34 and CD3 in one run, again with the ability to choose between the three panel options described herein in FIG. 9.

### Test Method

The AQUIOS CL Flow Cytometer is one system that can be used to implement the methods described herein and is a quantitative automated analyzer that performs the STEM diagnostic applications in a “no-wash” sample preparation process. Since this system is intended to be an automated analyzer with hands-off processing of samples from specimen introduction to results reports, it is referred to as a “Load & Go” flow cytometer. The AQUIOS System Software and AQUIOS STEM Tests and Quality Control Reagents do not require user verification of standardization of light scatter, and fluorescence intensities or verification of color compensation settings.

Automatic operation is initiated by creating requests and loading a cassette containing specimen tubes in the autoloader or a specimen tube in the Single-tube Loader. Samples are automatically processed according to these requests. The sample is stained and incubated, the red blood cells are lysed using the AQUIOS STEM Lysing solution. The white blood cells are analyzed on the AQUIOS CL Flow Cytometry system with the AQUIOS STEM Tests. STEM sample preparation is optimized to operate using barcoded 96-deep well plates with conical-shaped, deep wells. Each well holds up to 600 μL.

The AQUIOS STEM system can utilize up to two kits: AQUIOS STEM kit alone or in combination with AQUIOS STEM ALLO-CD3 kit. AQUIOS STEM-Kit reagents consist of a CD45-FITC/CD34-PE murine monoclonal antibody reagent, a corresponding negative control (CD45-FITC/CD34-CTRL), an absolute count reagent (AQUIOS STEM-Count Fluorospheres), a cell viability reagent (7-AAD), and a ready-to-use lysing reagent. AQUIOS STEM Allo-CD3 Kit is an optional reagent kit for the simultaneous enumeration of CD3+ T cells and CD34+ cells in sample material from allogeneic donors. The kit contains CD3-PC7 as well as an appropriate negative control.

AQUIOS STEM Tests are using the AQUIOS STEM Kit reagents containing monoclonal antibodies for the simultaneous identification and enumeration of viable absolute count of CD34+ HPC/μL, viable absolute count of CD45+/μL, and percentage of viable CD34+ HPC. Three AQUIOS STEM tests are available as part of the AQUIOS STEM menu (FIG. 9): stem panel: run tests in duplicate plus negative control (3 wells); stem duplicate: run tests in duplicate (no negative control; 2 wells); stem single: single test (1 well).

AQUIOS STEM ALLO Tests are using the AQUIOS STEM Kit reagent in combination with the AQUIOS STEM ALLO-CD3 Kit reagents. The combination of the two kits contains monoclonal antibodies and allows simultaneous identification and enumeration viable absolute count of CD34+HPC/μL, viable absolute count of CD45+/μL, viable absolute count of viable CD3+/μL and percentage of viable CD34+HPC. Three AQUIOS STEM ALLO tests are available (FIG. 9): STEM ALLO panel: run tests in duplicate plus negative control (3 wells); STEM ALLO duplicate: run tests in duplicate (no negative control; 2 wells); STEM ALLO single: single test (1 well).

All test use 43 μL of sample stained with 13 μL of each reagent (monoclonal antibodies and viability dye). After 15 minutes of incubation, the sample is lysed using 430 μL Lysing Reagent, then AQUIOS STEM-Count is added after the lyse incubation of approximately 15-minutes. The sample is then mixed and aspirated for analysis. For AQUIOS STEM/STEM ALLO Duplicate or AQUIOS STEM/STEM ALLO Panel, 2 or 3 wells will be used respectively.

### Purpose and Scope of the Study

As part of product characterization, this study was conducted to compare the equivalent performance of viable CD34+ absolute counts and CD3+ T cell absolute counts across the analysis options summarized in FIG. 9.

40 fresh apheresis samples were collected and analyzed. In order to assess equivalence between testing options for CD34+ enumeration, the analysis option “STEM Panel” (CD34 in duplicate plus negative control) was used as a reference for the two additional analysis options “STEM Duplicate” (CD34 in duplicate without negative control) and “STEM Single” (single test).

The same test scheme was performed for CD34+ and CD3+ enumeration with the analysis options that can include CD3 as additional marker, by using the analysis protocols “STEM ALLO Panel” (CD34 and CD3 in duplicate plus negative control) as a reference for the two additional analysis options “STEM ALLO Duplicate” (CD34 and CD3 in duplicate without negative control) and “STEM ALLO Single” (single test for CD34 and CD3).

The study was performed according to CLSI EP09c: Measurement Procedure Comparison and Bias Estimation Using Patient Samples; Approved Guidelines—Third Edition. Samples were analyzed using the AQUIOS CL flow cytometer equipped with AQUIOS STEM Software and AQUIOS STEM System reagents.

### Test Design

Daily startup and shutdown of the AQUIOS CL instrument used in the study were performed according to the manufacturer's Instructions for Use. Maintenance activities were recorded in the maintenance Log.

Daily quality control (QC) and process control runs were performed with Flow-Check Fluorospheres and AQUIOS STEM CD34 Control Cells in order to confirm the alignment of the instrument and to ensure performance within assigned assay values.

Apheresis samples used for the study were collected with ACD-A as anticoagulant and stored at 2-8° C. until use. Samples were either used undiluted if the white blood cell count was below 30,000 cells/μL, or otherwise were diluted in PBS/6% bovine serum albumin (BSA) to a white blood cell concentration not exceeding 30,000 cells/μL. Individual aliquots of each apheresis sample were used for a total of 12 runs according to the testing options summarized in FIG. 1. Hemolyzed specimens and specimens containing visible clots were excluded.

Data analysis was performed according to CLSI EP09c by using Weighted Deming regression. The upper and lower 95% confidence limits of bias estimated were calculated based on standard error of bias and compared to the manufacturer's acceptance limits (specifications).

### Results

### AQUIOS STEM System Test Option Equivalence for CD34+ Absolute Counts

The outcome of a hematopoietic progenitor cell transplantation depends on the successful infusion of a sufficient number of live CD34+ cells in order to reconstitute hematopoiesis in the recipient, so that the accurate determination of CD34+ cells by flow cytometry is a key component within the transplantation procedure.

In contrast to other solution for CD34+ enumeration, the AQUIOS STEM System offers a total of 6 different protocols for CD34+ enumeration, either with or without the optional analysis of CD3+ T cells (FIG. 9). In order to demonstrate equivalence between these different testing options, aliquots from the same samples were run with the different options and analyzed for correlation.

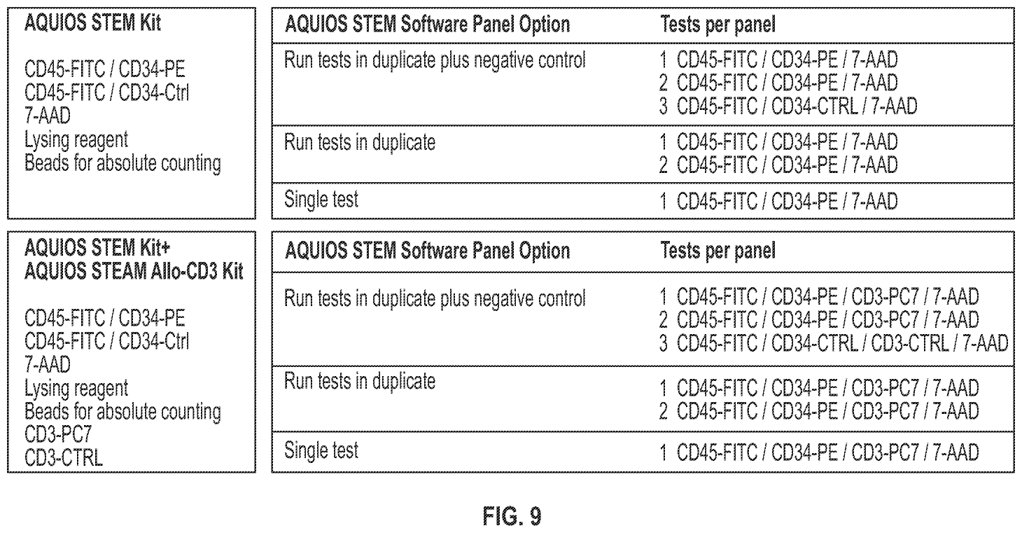

In a first set of experiments, the absolute number of CD34+ cells/μL was assessed by running apheresis samples with the three analysis protocol options that do not include CD3 (STEM Panel, STEM Duplicate, STEM Single) in order to demonstrate the equivalence for CD34 enumeration between these three options (FIGS. 10A-10C). For the comparison of analysis options where tests are performed in duplicate (STEM Panel vs. STEM Duplicate, FIG. 10A), the mean of both runs was used for analysis. When comparing test options that include duplicate runs against single run test options (STEM Panel vs. STEM Single (FIG. 10B) and STEM Duplicate vs. STEM Single (FIG. 10C)), the result of the first run was used as reference. For all regression pairs analyzed, the equivalent performance of viable CD34+ cell absolute counts across analysis options was demonstrated to be within defined acceptance criteria.

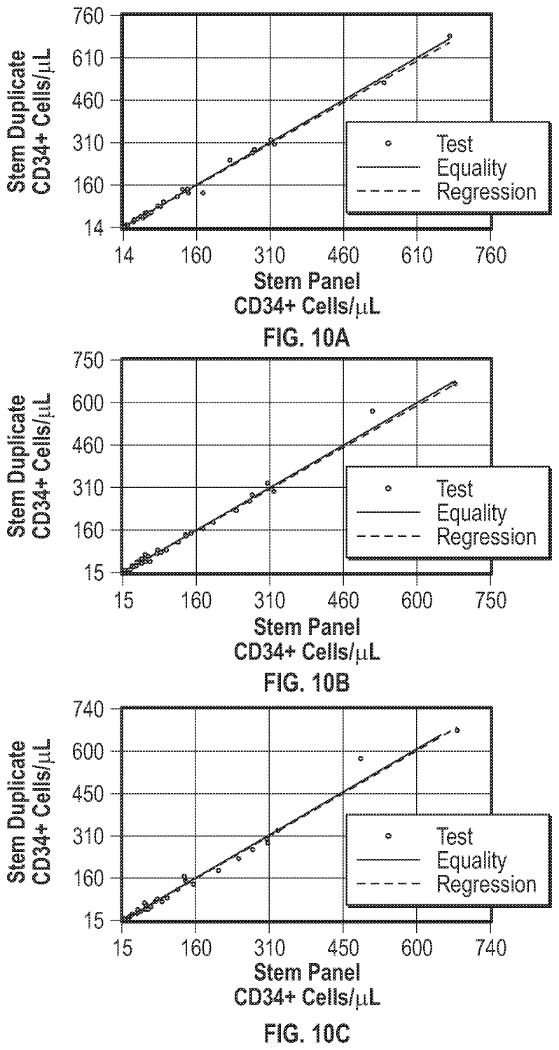

In a second set of experiments, the number of CD34+ cells were analyzed by using the three analysis protocols that are intended for the parallel enumeration of CD34+ and CD3+ cells; all other criteria were kept identical (FIGS. 11A-11C). Again, for all regression pairs analyzed, the equivalent performance of viable CD34+ cell absolute counts across analysis options was demonstrated to be within defined acceptance criteria also for the analysis protocols with CD3.

In summary, all six analysis options for CD34+ enumeration, either with or without CD3, provided equivalent results for the correlation pairs analyzed.

### AQUIOS STEM System Test Option Equivalence for the Enumeration of Residual CD3+ T Cells

In cases of hematological diseases or non-malignant dysfunction of the hematopoietic system, CD34+ cells are collected from the peripheral blood (PB), bone marrow (BM) or cord blood (CB) of non-self (allogeneic) donors. Like in autologous settings, where the donor and the recipient are the same person, mobilized PB today is the most commonly used source for CD34+ stem and progenitor cells in allogeneic transplant schemes.

The use of CD34+ cells from mobilized peripheral blood has the advantage of a relatively rapid recovery of hematopoiesis after transplantation but goes along with an increased risk of acute Graft versus Host Disease (aGvHD) due to a higher number of circulating T cells. As acute and chronic GvHD affects approximately 30-40% of patients who undergo allogeneic transplantation, and donor T cells are recognized as playing a central role in mediating aGvDH, the enumeration of CD3+ T cells in the graft together with CD34+ cells has become standard practice in many laboratories.

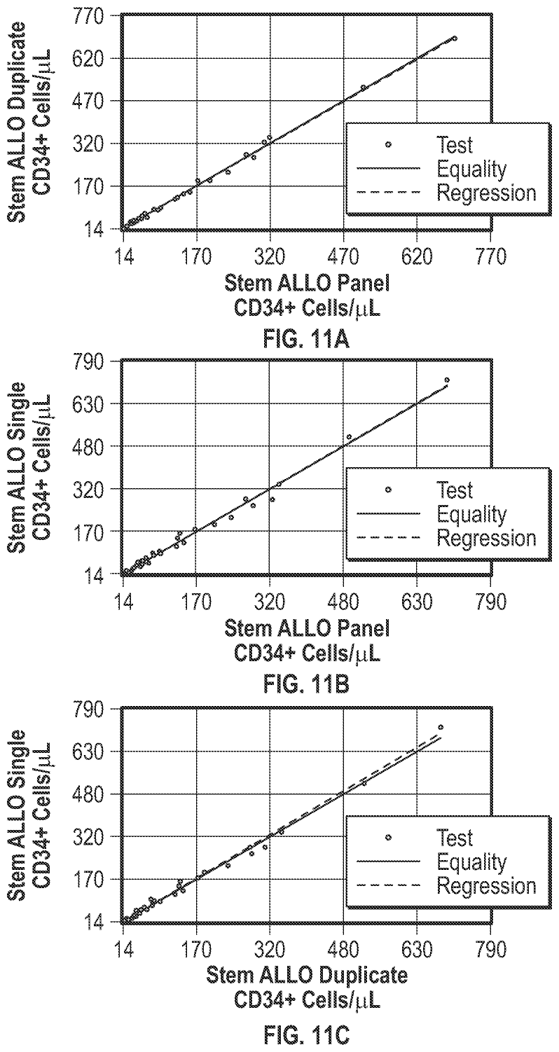

In addition to the standard test protocols for CD34+ enumeration, the AQUIOS STEM System provides the option to analyze CD3+ T cells together with CD34+ cells in one run. In order to ensure test option equivalence not only for CD34+ analysis but also for CD3+ enumeration, a third set of experiments was performed that compared the outcome of CD3+ enumeration for the 3 analysis options described herein (FIGS. 12A-12C). As for CD34+, the equivalent performance of viable CD3+ cell absolute counts across analysis options was demonstrated to be within defined acceptance criteria for all regression pairs analyzed.

### Conclusion

The AQUIOS STEM System is a modular approach for the automated analysis of CD34+ hematopoietic stem and progenitor cells on the AQUIOS CL Flow Cytometer and is intended to overcome the limitations of currently available solutions in terms of panel and assay flexibility.

The present study addressed the question in how far the test options offered by the system result in equivalent data for CD34+ and CD3+ absolute counts, in order to exclude potential bias by choosing one option over the other.

In summary, it was possible to demonstrate equivalence of data for all regression pairs analyzed, and the equivalent performance of viable CD34+ cell absolute counts and viable CD3+ absolute counts across analysis options was demonstrated to be within defined acceptance criteria.

### Example 2

### Workflow Comparison Between AQUIOS STEM System and its Predicate Method, the FC500 Stem-Kit

### Background

Today more than 50,000 hematopoietic stem and progenitor cell (HPC) transplantations are carried out annually worldwide for the treatment of several hematological malignancies as well as for non-hematological indications.

Clinical laboratories rely on commercially available IVD solutions for CD34+ HPC enumeration in order to avoid time and resource consuming validation of user-defined tests. Most reagent kits and software packages were developed as a response to the 1996 and 1998 ISHAGE Guidelines but were since not updated to meet the evolving demands of diagnostic laboratories, especially in terms of automation capability. In hemato-oncological laboratories, HPC samples often arrive as emergency (STAT) samples in the laboratory and require immediate attention, disrupting the routine workflow. Any issues with these samples duplicate efforts and thus increase the potential for human error. For these laboratories, it would be desirable to integrate HPC samples into the normal workflow, ideally in a way that minimizes the risk for sampling mistakes or other issues, as the analysis of CD34+ HPC is time critical.

This example compares the workflow of the newly developed AQUIOS STEM System for automated CD34+ enumeration against its predicate method, the Stem-Kit for the FC500 flow cytometer (both Beckman Coulter, Inc.), in terms of turnaround time and operator hands-on time.

### Overview of a Traditional Flow Cytometry Workflow

Many traditional flow cytometry solutions for CD34+ enumeration, such as the Beckman Coulter Stem-Kit for FC500, require manual preparation of samples, manual creation of worklists, manual data review, and manual tabulation of numerical data. This workflow can result in longer run times, more hands-on time, and requires more experienced operators.

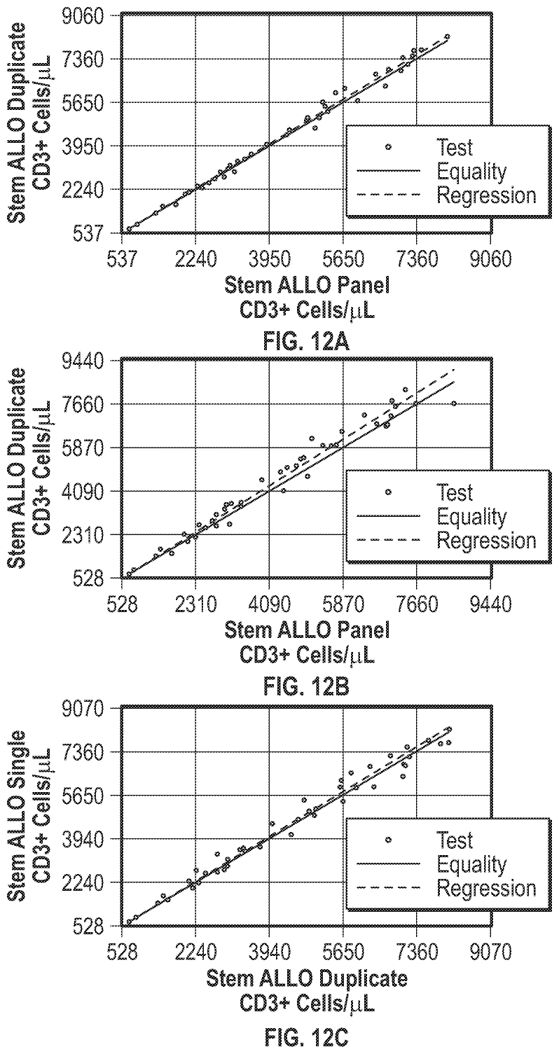

In order to perform a CD34+ enumeration test with the predicate method (Stem-Kit on FC500), the operator is required to manually check for instrument alignment, prepare controls to verify and adjust fluorochrome spillover (compensation), prepare and run process controls, and finally prepare and run the actual patient/donor samples. Resulting data needs to be verified and manually transferred to the laboratory information system (LIS). Reagent and quality control (QC) logbooks are typically kept manually. See FIG. 13 for a representative manual flow cytometry workflow.

### Overview of the AQUIOS CL Flow Cytometer Workflow

The AQUIOS CL is an automated system that performs the majority of the preparatory steps leading to a test result, so that most manual preparation steps are eliminated.

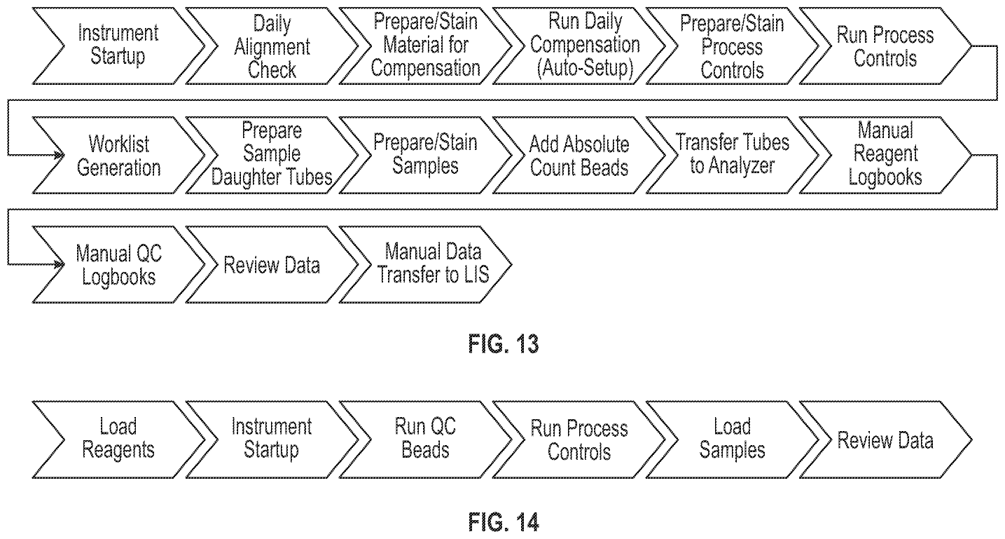

For the AQUIOS STEM System for CD34+ enumeration on AQUIOS, the operator needs to load the reagents on the system once, start up the instrument, run QC samples, and is then ready to load and analyze patient samples throughout the remaining workday (FIG. 14).

### Comparison Protocol

The purpose of this case study was, among other things, to evaluate the AQUIOS CL Flow Cytometer design for CD34+ enumeration, and to model its resultant workflow through comparison with the Beckman Coulter Stem-Kit on FC500 (predicate method). This case study estimates the following workflow parameters:

Turnaround time—For alternate systems, the time duration begins with the first sample preparation step and ends with completed test results. For the AQUIOS CL system, time duration begins with the sample placed in the autoloader and ends with the completed test result for the last sample being displayed. For both systems, the time does not include QC.

Operator hands-on time—The time required by the user to physically perform the steps listed in the provided test procedures according to the manufacturer's Instructions for Use.

Time points were captured by videotaping sample preparation and analysis processes and modeled using an in-house workflow analysis software.

### Results

Sample processing turnaround time and operator hands-on time This test case consisted of scenarios with either 1 sample or a batch of 10 samples of mobilized peripheral blood, prepared in duplicate plus negative control. The AQUIOS STEM System was compared directly to its predicate method, the Stem-Kit on FC500. Data are shown for tests that consist of duplicate runs plus negative control.

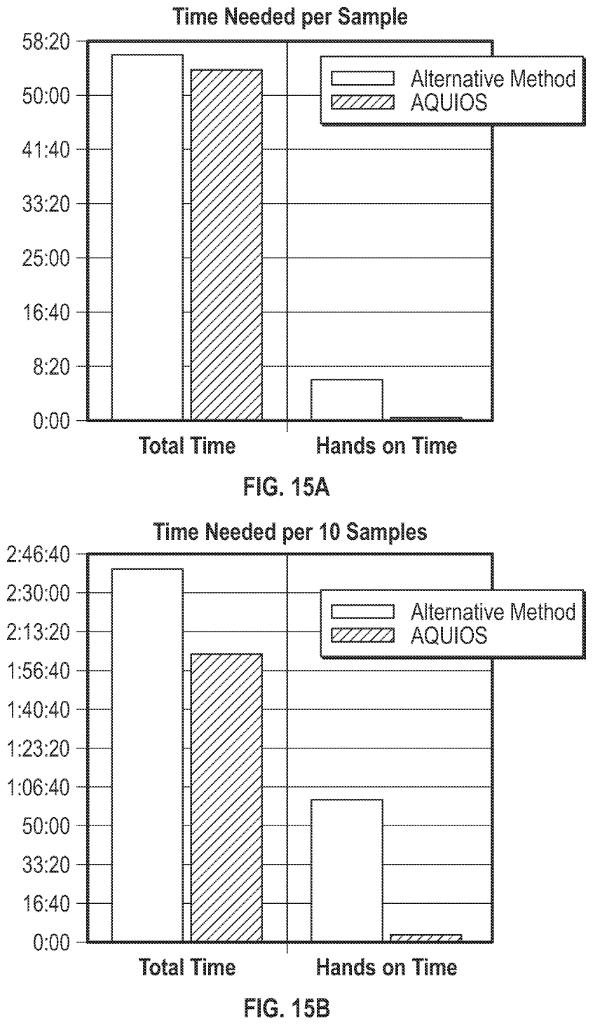

For 1 specimen sample, time to result from sample preparation to result generation did not show a major difference (56:19 minutes for the predicate method vs. 53:54 minutes for AQUIOS; FIG. 15A), as the process steps are mainly determined by the incubation times with the antibodies and red blood cell lysis reagent.

However, operator hands-on time could be reduced by 95%, from 6:23 minutes for the predicate vs. 0:20 minutes on AQUIOS (FIG. 15A).

For a batch of 10 specimen samples (an average daily workload in a mid-sized stem cell transplantation center), these differences become more prominent. Time to result from specimen preparation to result generation went down from 2:40:29 hours for the predicate method to 2:03:54 hours for AQUIOS (FIG. 15B), with operator hands-on time being reduced from 1:01:13 hours for the predicate method to 3:20 minutes for the AQUIOS STEM System (FIG. 15B).

As also for a manual process certain steps can be performed in parallel (pipetting the next sample during the incubation time of the first sample, etc.), the overall time for 10 samples is shorter than the sum of 10 times one sample.

Assuming 10 samples per workday over the period of one year, the AQUIOS STEM System saves a lab hundreds of hours of valuable tech time each year. In addition, the faster time to result when running an average workload of 10 samples per day supports time-critical assays such as CD34+ enumeration.

### Process Time and Operator Hands-On Time for Quality Control (QC) Procedures

In a regulated environment, system performance needs to be controlled with appropriate quality and process controls for both the instrument settings as well as the test to be run. For both the Stem-Kit on FC500 and AQUIOS, daily verification of the flow cytometers optical alignment and fluidics system is performed with Flow-Check Fluorospheres, an assayed suspension of fluorospheres (fluorescent microspheres). As process controls for the verification of the analytical parameters of interest, the predicate method uses lyophilized CD34+ cells that are added to a normal blood sample (Stem-Trol Cells), while the AQUIOS STEM System uses liquid preparations of stabilized human leukocytes (lymphocytes, monocytes and granulocytes) and erythrocytes that have lysing, light scatter, antigen expression and antibody staining properties representative of those found in human whole blood specimens (AQUIOS STEM CD34 Control Cells).

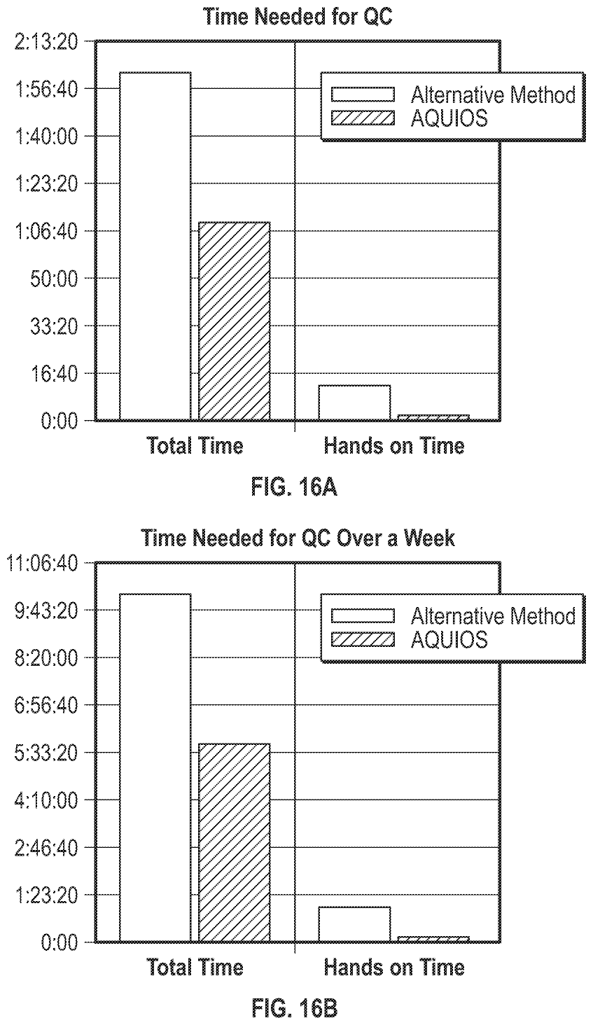

The total time needed for QC procedures related to CD34+ enumeration was reduced from 2:02:22 hours for the predicate method to 1:09:44 hours on AQUIOS. Of note, QC needs to be completed before patient/donor samples can be run, which leads to a time saving of 52:38 minutes per day (FIG. 16A). For a 5-day work week, this sums up to approx. 4.5 hours only for the QC procedures (FIG. 16B).

Operator hands on time for QC procedures is reduced from 12:22 minutes (predicate) to 1:51 minutes (AQUIOS) per day (FIG. 16A), resulting in a total hands-on time saving for QC processes of 52 minutes per week (FIG. 16B).

### Conclusion

In all test case scenarios, the AQUIOS STEM System required substantially less hands-on time than the predicate method. For a typical 5-day work week with 10 specimen samples per day, AQUIOS STEM System reduces manual workload by approx. 6 hours, allowing the lab to allocate resources more efficiently. In addition, AQUIOS STEM reduces the overall turnaround time from sample preparation to patient result, which is essential for a time-critical test such as CD34+ enumeration

The AQUIOS STEM System is a quantitative automated solution that performs the enumeration of CD34+ hematopoietic stem and progenitor cells in a “no-wash” sample preparation process. Since this system is intended to be an automated analyzer with hands-off processing of samples from specimen introduction to results reports, it is referred to as a Load & Go flow cytometer. The automation features differentiate AQUIOS STEM System from alternative methods (including its predicate method, i.e. FC500 with Stem-Kit), where many process steps need to be performed manually. The all-in-one approach of the AQUIOS STEM System is intended to assist in time savings and to help increase the workflow efficiency of a modern laboratory.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the disclosure, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” as used herein refers to less than about 30%, 25%, 20%, 15%, 10%, 5%, 3%, 2%, 1%, 0.5%, 0.1%, 0.05%, 0.001%, or at less than about 0.0005% or less or about 0% or 0%.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

1. An automated flow cytometric method for the analysis and enumeration of at least one of hematopoietic stem cells, hematopoietic progenitor cells, and T-cells, the method comprising:
placing one or more blood samples into one or more receptacles of a sample plate located in a sample preparation area of a flow cytometer;
treating the one or more receptacles with at least one reagent to obtain at least one first composition, at least one of the at least one reagent being at least one imaging reagent comprising recognition elements specific for markers on at least one of the hematopoietic stem cells, the hematopoietic progenitor cells, and T-cells;
incubating the at least one first composition for a time period sufficient for binding of the at least one imaging reagent comprising recognition elements specific for the markers on the at least one of the hematopoietic stem cells, hematopoietic progenitor cells, and T-cells to give at least one second composition;
treating the at least one second composition with a lysing reagent to give at least one third composition; and
analyzing the at least one third composition by flow cytometry to obtain at least one of a viable and total hematopoietic stem cell count viable and total progenitor cell count, and viable and total T-cell count in the one or more blood samples.

2. The method of claim 1, wherein the method is conducted with duplicate blood samples.

3. The method of claim 2, wherein the method is conducted with a negative control.

4. The method of claim 3, wherein the negative control is for CD34.

5. The method of claim 3, wherein the negative control is for CD3.

6. The method of claim 1, wherein the markers are at least one of CD45, CD3, and CD34.

7. The method of claim 1, wherein the at least one imaging reagent comprises a fluorescent reporter.

8. The method of claim 1, wherein the at least one imaging reagent comprises an FITC, PE or PC7 fluorescent reporter.

9. The method of claim 1, wherein the first composition further comprises a viability dye.

10. The method of claim 9, wherein the viability dye is 7-aminoactinomycin D (7-AAD).

11. The method of claim 1, wherein viable and total hematopoietic stem cell counts, viable and total progenitor cell counts, and viable and total T-cell counts are obtained.

12. The method of claim 1, wherein the sample plate is a 96-well microtiter plate.

13. The method of claim 1, wherein the treating the one or more receptacles with at least one reagent to obtain at least one first composition is performed by an automated pipettor configured to deliver predetermined volumes for the at least one reagent.

14. The method of claim 1, wherein the incubating is automated to incubate the at least one first composition for a predetermined time period sufficient for binding of the at least one imaging reagent comprising recognition elements specific for the at least one of the hematopoietic stem cells, hematopoietic progenitor cells, and T-cells to give at least one second composition.

15. An automated flow cytometric method for the analysis and enumeration of at least one of hematopoietic stem cells, hematopoietic progenitor cells, and T-cells, the method comprising:
placing a first blood sample into a first receptacle of the sample plate;
treating the first receptacle with at least one reagent to obtain a first composition f¹ and incubating first composition f¹ to give second composition s¹, at least one of the at least one reagent being at least one imaging reagent comprising recognition elements specific for markers on at least one of the hematopoietic stem cells, hematopoietic progenitor cells, and T-cells;
while first composition f¹ is incubating, placing a second blood sample into a second receptacle of the sample plate and treating the second receptacle with at least one reagent, at least one of the at least one reagent being at least one imaging reagent comprising recognition elements specific for markers on at least one of hematopoietic stem cells, hematopoietic progenitor cells, and T-cells, to obtain a first composition f² and incubating first composition f² to give second composition s²;
treating second compositions s¹ and s² with a lysing reagent to give third compositions t¹ and t²; and
analyzing third compositions t¹ and t² by flow cytometry to obtain at least one of a viable and total hematopoietic stem cell count, viable and total progenitor cell count, and viable and total T-cell count.

16. The method of claim 14, further comprising preparing a negative control.

17. The method of claim 15, wherein the negative control is prepared before the first composition f¹.

18. The method of claim 15, wherein the negative control is prepared after the first composition f¹.

19. The method of claim 15, wherein the negative control is prepared after the first composition f².
